(19) United States
(12) Patent Application Publication
(10) Pub. No.: US 2023/0011197 A1
(43) Pub. Date: Jan. 12, 2023

(54) DEVICE AND METHOD FOR STIMULATING SKIN CELLS USING A MICROCURRENT

(71) Applicant: L'OREAL, Paris (FR)

(72) Inventors: Carine BALTENNECK, AULNAY-SOUS-BOIS (FR); Sarah HUBERT, AULNAY-SOUS-BOIS (FR); Yegor DOMANOV, AULNAY-SOUS-BOIS (FR)

(21) Appl. No.: 17/784,829

(22) PCT Filed: Dec. 14, 2020
(86) PCT No.: PCT/EP2020/085913

§ 371 Date: Jun. 13, 2022

(30) Foreign Application Priority Data
Dec. 18, 2019 (FR) FR1914766

(51) Int. Cl. A61N 1/32; A61N 1/36

(52) U.S. Cl. CPC A61N 1/328; A61N 1/36034; A61N 1/36031

(57) ABSTRACT

A device for electrically stimulating skin, in particular for stimulating the activity of fibroblasts, including an electronic circuit for generating a sawtooth biphasic or cyclic alternating electric current; at least two electrodes connected to the electronic circuit, which are configured so as to be applied to the skin in order to allow the electric current generated to pass therethrough.

### TECHNICAL FIELD

The present invention relates to devices for carrying out a treatment, in particular a cosmetic or dermatological treatment, of the skin, in particular that of the body or of the face, and to methods implemented with these devices.

### PRIOR ART

The application of electric currents to various parts of the human body to obtain different cosmetic or therapeutic outcomes has already been proposed, in particular, the application of an electric current to the skin is known to stimulate fibroblasts, which are responsible for the synthesis, breakdown and remodeling of dermal tissue, and promote the secretion of proteins, like collagen for example, allowing in particular the regeneration of the skin.

US 2003/191508, U.S. Pat. No. 6,684,107 and US 2008/195181 describe cosmetic treatment devices that deliver an electric current mainly in the form of a square or rectangular wave.

U.S. Pat. No. 9,968,773 and US 2003/233137 describe therapeutic treatment devices for improving cicatrization by delivering an electric current mainly in the form of a square or rectangular wave to the skin.

Furthermore, electric currents are applied to the skin in iontophoresis methods for promoting the penetration of molecules.

In particular, subjects of WO 18 127 433, EP 3 459 588 and EP 3 459 589 are iontophoresis methods comprising a step of applying an electric current which may be in the form of a square, rectangular, pointed, trapezoidal, triangular, or sawtooth wave, or a combination of these forms..

### DISCLOSURE OF THE INVENTION

It is advantageous to further refine devices that promote the regeneration of the skin without adversely affecting it.

There is in particular a need for a device for treating, in particular cosmetically or dermatologically, the skin, in particular of the body or of the face, using an electric current, that is highly effective and may be used with comfort and in complete safety.

### SUMMARY OF THE INVENTION

The invention aims to meet this need and achieves this, according to one of the aspects thereof, by virtue of a device for electrically stimulating skin, in particular for stimulating the activity of fibroblasts, comprising:
an electronic circuit for generating a sawtooth biphasic or cyclic alternating electric current;
at least two electrodes connected to the electronic circuit, which are configured so as to be applied to the skin in order to allow the electric current thus generated to pass therethrough.

By passing through the skin located between the electrodes, the electric current stimulates the fibroblasts, remodeling the dermis and affecting the appearance of the skin, in. particular by decreasing the number of wrinkles, and by making it firmer and smoother.

Surprisingly, the applicant has discovered that the sawtooth waveform of the electric current allows the fibroblasts to be activated more effectively, and therefore the remodeling of the dermis to be accelerated, improving the appearance and consistency of the skin. Stimulating the skin by means of a device according to the invention makes it possible, in particular, to prevent, decrease, or even reverse the effects of aging on the skin at the level of the structural and physical properties thereof, and in particular to act on the suppleness, tone, firmness, elasticity, uniformity, skin. slackening, or else improve blood circulation. The invention makes it possible to act on the synthesis and secretion, by the fibroblasts, of molecules involved in the homeostasis of the dermis and of the skin .in general; these molecules are, for example, signaling factors, dermal fibers or precursors thereof, enzymes or activators/inhibitors thereof, etc. Modulating the synthesis and secretion of these molecules may have an effect on the reorganization (or remodeling of the extracellular matrix in the dermis, with positive consequences for skin tone.

Electric Current

The electric current generated has, according to the invention, a sawtooth waveform.

A sawtooth signal is a preferably periodic signal that reproduces a succession of elementary patterns, each composed of a substantially monotonic variation of the electric current in one direction followed by a substantially monotonic variation of the electric current in the opposite direction.

Thus, for each substantially monotonic variation of the electric current intensity delivered by the electronic circuit, dlfdt is substantially constant, in particular constant to within +/−20%, more preferably to within +/−10%, even more preferably to within +/−5%. The first time derivative, dI/dt, exhibits a discontinuity that becomes more marked as the transition between the two monotonic variations becomes shorter.

An elementary pattern comprises in particular a rising edge, corresponding to an increase in the intensity of the electric current in terms of absolute value, and a falling edge, corresponding to a decrease in the intensity of the electric current in terms of absolute value. Preferably, these variations within an elementary pattern are linear.

The electric current advantageously exhibits changes in polarity, the elementary patterns therefore being of positive or negative polarity. The changes in polarity preferably take place periodically, for example after each elementary pattern, which gives an alternating current, and/or after a predefined duration, in the case of a biphasic current.

In general, the frequency of the electric current may be between 1 Hz and 1000 Hz, more preferably between 50 Hz and 200 Hz, even more preferably about 100 Hz. This frequency corresponds to the frequency of repetition of the elementary patterns.

Preferably, the peak intensity of the electric current is constant in terms of absolute value.

Rising Edge

A rising edge of an elementary pattern may comprise an increase in the intensity of the electric current per second and in terms of absolute value of between 0.001 A and 2.5 A, more preferably of between 0.01 A and 0.3 A, even more preferably of about 0.05 A.

As a variant, the rising edge of an elementary pattern comprises an increase in the intensity of the electric current per second and in terms of absolute value of between 0.05 A and 100 A. more preferably of between 0.5 A and 10 A, even more preferably of about 2.1 A.

The device according to the invention may be configured such that a rising edge of an elementary pattern extends over a duration of between 1/2000 and ½ of the duration of the elementary pattern, more preferably over a duration of between 1/200 and of the duration of the elementary pattern, even more preferably over a duration of about 1/42 of the duration of the elementary pattern.

As a variant, and preferably, the device is configured such that a rising edge of an elementary pattern extends over a duration of between ½ and 1999/2000 of the duration of the elementary pattern, more preferably over a duration of between ¾ and 199/200 of the duration of the elementary pattern, even more preferably over a duration of about 41/42 of the duration of the elementary pattern.

Falling Edge

A falling edge of an elementary pattern may comprise a decrease in the intensity of the electric current per second and in terms of absolute value of between 0.001 A and 2.5 A, more preferably of between 0.01 A and 0.3 A, even more preferably of about 0.05 A. Preferably, a falling edge comprising a decrease in intensity of the electric current per second and in terms of absolute value of between 0.001 A and 2.5 A, more preferably of between 0.01 A and 0.3 A, even more preferably of about 0.05 A, is preceded by a rising edge comprising an increase in the intensity of the electric current per second and in terms of absolute value of between 0.05 A and 100 A, more preferably of between 0.5 A and 10 A, even more preferably of about 2.1 A.

As a variant, a falling edge of an elementary pattern comprises a decrease in the intensity of the electric current per second and in terms of absolute value of between 0.05 A and 100 A, more preferably of between 0.5 A and 10 A, even more preferably of about 2.1 A. Preferably, a falling edge comprising a decrease in intensity of the electric current per second and in terms of absolute value of between 0.05 A and 100 A, more preferably of between 0.5 A and 10 A, even more preferably of about 2.1 A, is preceded by a rising edge comprising an increase in the intensity of the electric current per second and in terms of absolute value of between 0..001 A and 2.5 A, more preferably of between 0.01 A and 0.3 A, even more preferably of about 0.05 A.

The device may be configured such that a falling edge of an elementary pattern extends over a duration of between ½ and 1999/2000 of the duration of the elementary pattern, more preferably over a duration of between ¾ and 199/200 of the duration of the elementary pattern, even more preferably over a duration of about 41/42 of the duration of the elementary pattern.

As a variant, and preferably, the device is configured such that a falling edge of an elementary pattern extends over a duration of between 1/2000 and ½ of the duration of the elementary pattern, more preferably over a duration of between 1/200 and ¼ of the duration of the elementary pattern, even more preferably over a duration of about 1/42 of the duration of the elementary pattern. Thus, the falling edge has a very steep slope.

Preferably, an elementary pattern comprises a falling edge that extends over a duration of between ½ and 1999/2000 of the duration of an elementary pattern, more preferably over a duration of between ¾ and 199/200 of the duration of the elementary pattern, even more preferably over a duration of about 41/42 of the duration of the elementary pattern and a rising edge that extends over a duration of between 1/2000 and ¼ of the duration of the elementary pattern, more preferably over a duration of between 1/200 and ¼ of the duration of the elementary pattern, even more preferably over a duration of about 1/42, or a falling edge that extends over a duration of between 1/2000 and ¼ of the duration of the elementary pattern, even more preferably over a duration of between 1/200 and ¼ of the duration of the elementary pattern, even more preferably over a duration of about 1/42 and a rising edge that extends over a duration of between ½ and 1999/2000 of the duration of an elementary pattern, more preferably over a duration of between ¾ and 199/200 of the duration of the elementary pattern, even more preferably over a duration of about 41/42 of the duration of the elementary pattern.

Elementary Pattern

The rising edge and the falling edge of an elementary pattern may be symmetrical. Preferably, the rising edge and the falling edge of an elementary pattern are asymmetric.

Preferably, the device is configured such that a rising edge of an elementary pattern extends over a longer duration than a falling edge of the elementary pattern. Thus, the slope in terms of absolute value of the rising edge, |dI/dt|, is, preferably, less than that of the falling edge.

The electronic circuit is, preferably, arranged such that the electric current has elementary patterns that extend over a duration of between 1 ms and 100 ms, more preferably of between 5 ms and 20 ms, even more preferably of about 10 ms.

Two elementary patterns of an electric current exhibiting a sawtooth waveform and being of opposite polarity may be symmetric or asymmetric. Preferably, they are asymmetric.

Cyclic Alternating Current

The device may be configured to generate a cyclic alternating electric current.

A cyclic alternating current comprises a succession of elementary patterns, such that a change in polarity takes place after each elementary pattern.

Preferably, the change in polarity occurs periodically. Each elementary pattern then extends over the same duration,

Biphasic Current

Preferably, le device is configured to generate a biphasic electric current.

A biphasic electric current comprises two successive cycles of opposite polarities. Each sawtooth cycle extends over at least two elementary patterns. Preferably, each cycle comprises between 3 and 100 elementary patterns, more preferably between 5 and 20 elementary patterns, even more preferably about 10 elementary patterns.

Two cycles of opposite polarities may comprise an equal or different number of elementary patterns, Preferably, a cycle of a certain polarity comprises the same number of elementary patterns as the cycle of opposite polarity.

Two cycles of opposite polarities may extend over different durations.

Preferably, the change in polarity occurs periodically. Thus, two cycles of the same polarity and/or of opposite polarities extend over the same duration,

The cycles of a biphasic electric current may be asymmetric. The elementary patterns of a cycle of a certain polarity may be different from the elementary patterns of a cycle of opposite polarity, in terms of rising edge, falling edge, duration, amplitude of the elementary patterns in terms of absolute value and/or number of elementary patterns.

Preferably, cycles of opposite polarities comprise asymmetric elementary patterns. In particular, the slope in terms of absolute value of a rising edge of positive polarity may be substantially equal to the slope in terms of absolute value of a falling edge of negative polarity and/or the slope in terms of absolute value of a falling edge of positive polarity may be substantially equal to the slope in terms of absolute value of a rising edge of negative polarity. What should be understood by “substantially equal” is of the same order of magnitude, and more preferably equal to within +/−50%, even more preferably equal to within +/−20%.

However, the amplitude of the elementary patterns of the cycles of a certain polarity is preferably equal, in terms of absolute value, to the amplitude of the elementary patterns of the cycles of opposite polarity. In general, the current generated is preferably asymmetric.

Electrodes

The electrodes may be used statically or otherwise on the skin, generally being in contact with the skin.

Thus, in one embodiment, the electrodes are to be attached to the skin, being for example in the form of a mask with a plurality of electrodes printed or distributed in another way over the surface of the mask.

As a variant, the electrodes are moved over the skin, by being for example slid thereover, the treatment taking place dynamically in order to distribute the current and treat different regions.

The electrodes may have an area of contact with the skin that is flat or rounded, in particular spherical, paraboloidal or ellipsoidal in shape. Additionally, the electrodes may be rotary, taking for example a ball or roller form, thus allowing the electrodes to be moved easily over the skin.

The electrodes may be flexible or rigid.

Preferably, the electrodes are rigid and rounded in shape, or flexible and to be attached to the skin.

It is advantageous for each electrode to have an area of contact with the skin that is sufficiently large so as not to cause local heating of the skin due to a high electric current density, and/or discomfort due to a tingling sensation.

Preferably, each electrode thus has an area of contact with the skin that is greater than or equal to 1 cm².

The electrodes may have equal or different contact areas.

Preferably, the intensity of the current is such that the maximum (peak) electric current density delivered by the electrodes is between 0.1 mA/cm² and 2.5 mA/cm², more preferably between 0.3 mA/cm² and 1.5 mA/cm², even more preferably about 0.5 mA/cm².

Advantageously, a device according to the invention allows the application of a current of low intensity that is hardly perceptible, or even imperceptible to the user undergoing the treatment.

Voltage

The electronic circuit is preferably configured such that the maximum (peak) voltage between the electrodes without load is between 0.01 V and 45 V, more preferably between 0.1 V and 20 V. The voltage is advantageously limited, allowing the device according to the invention to be used safely,

The voltage is preferably adjusted according to the anatomical region being treated, the hydration of the region, and the potential presence of a conductive gel, in order to maintain a substantially constant electric current intensity,

Gel

The device according to the invention may comprise a conductive gel to be applied to the skin, in the region of application of the electrodes, allowing the electric current between the electrodes to be stabilized.

The gel may be an aqueous solution comprising for example sodium chloride and a gelling agent.

The gel may be a suitable care formulation, which does not comprise any cosmetic active agent intended to migrate through the skin.

The conductive gel may be without a cosmetic active agent such as for example humectant or moisturizing active agents, anti-aging active agents, for example depigmenting active agents, active agents acting on skin microcirculation or seboregulating active agents, in particular vitamin C and derivatives thereof, hyaluronic acid, ellagic acid, glycerol and derivatives thereof, urea and derivatives thereof, lactic acids, AHAs, BHAs, sodium pidolate, xylitol, serine, sodium lactate, ectoin and derivatives thereof, chitosan and derivatives thereof, collagen, plankton, arginine, vitamin CG, CP and 3-O ethyl vitamin C. alpha and beta arbutin, ferulic acid, lucinol and derivatives thereof, kojic acid, resorcinol. and derivatives thereof, tranexamic acid and derivatives thereof, gentisic acid, homogentisate, lipoic methyl, vitamin B3, linoleic acid and derivatives thereof, ceramides and homologs thereof, anti-glycation agents, NO-synthase inhibitors, agents for stimulating the synthesis of dermal or epidermal macromolecules and/or the breakdown thereof, agents for stimulating fibroblast and/or keratinocyte proliferation, agents for stimulating or decreasing keratinocyte differentiation, muscle relaxants and/or dermo-decontracting agents, or free-radical scavengers. This list is not limiting.

Housing

The electrodes may be borne by a housing accommodating the electronic circuit, and facilitating the handling of the electrical stimulation device according to the invention. As a variant, each electrode is connected to the housing via at least one electrical wire. Each electrode, connected to the housing by a wire, is flexible and to be attached to the skin.

In particular, the electrodes may be borne by a housing accommodating an electrical energy source, in particular at least one battery or cell. As a variant, the electrical energy source is outside the housing.

Preferably, the electrodes borne by the housing are rigid and rounded in shape.

The electrical stimulation device is advantageously of a size allowing it to be handled by one hand, facilitating the application of the electric current to the skin.

The device according to the invention may comprise a member for adjusting the density of the electric current flowing between the electrodes. This member may be manually actua.table. Alternatively, the device according to the invention may comprise automatic adjustment of the electric current density.

The electric current density (A/cm²) may thus be regulated so as to ensure the efficacy of the treatment and/or to limit painful sensations. The density of the electric current may be regulated manually by the user, the user being able to modify the intensity of the electric current at will, or else when a warning facility signals to them that this is necessary, or automatically, by virtue of a system for feedback-controlling the electric current density.

In particular, the invention may comprise the analysis, by a connected system, of information relating to the skin of the user.

Said information may in particular originate from electrical measurements taken with the electrodes, for example in order to automatically regulate the intensity of the electric current according to the skin of the user.

The connected system may be for example a computer, a smartphone, or a smartwatch, inter alia, which may in particular communicate with the device for electrically stimulating skin by virtue of a wireless connection, in particular via the Internet.

The electrical stimulation device may be arranged so as to take at least one electrical measurement on the skin of the user before electrically stimulating same. This measurement may be performed via the electrodes used for the electrical stimulation. The electrical stimulation that is performed after this measurement may take into account at least one parameter deduced from the measurement. Thus, the electrical stimulation may be adapted to the person being treated.

Method for Cosmetically Treating the Skin

The invention further relates, according to another aspect thereof, to a method for cosmetically treating a healthy skin, comprising the step of subjecting the skin to a sawtooth biphasic or cyclic alternating electric current, in particular delivered by means of a device according to the invention.

The method according to the invention may comprise the step of moving the electrodes during the treatment, allowing different skin regions, in particular on the body or on the face, to be stimulated more easily. The method may in particular stimulate fibroblasts.

Preferably, the one or more skin regions treated exhibit a lack of firmness, tone or elasticity or comprise at least one wrinkle. The one or more skin regions treated may comprise at least one microlesion, in particular a split, a crack, or an abrasion.

The method according to the invention may comprise the repeated stimulation of the skin by massaging one or more regions thereof, in particular by performing circular, linear or curving movements. The application of the electric current according to the invention may take place over a duration of up to about 40 minutes, more preferably over a duration of between 1 minute and 20 minutes, even more preferably over a duration of between 2 minutes and 10 minutes.

Preferably, the application of the electric current is repeated regularly, for example at least once a week, more preferably once or twice a day.

The electric current may be applied in the presence of a conductive gel that is without a cosmetic active agent, in particular such as humectant or moisturizing active agents, anti-aging active agents, for example depigmenting active agents, active agents acting on skin microcirculation or seboregulating active agents, in particular vitamin C and derivatives thereof, hyaluronic acid, or ellagic acid,

### BRIEF DESCRIPTION OF THE DRAWINGS

The invention may be understood better from reading the following detailed description of a non-limiting exemplary embodiment thereof and from examining the appended drawing, in which:

FIG. 1 is a block diagram of an electrical stimulation device according to the invention;

FIG. 2 schematically shows an exemplary housing;

FIG. 3 shows different forms of current, some according to the invention and others not;

FIG. 4 gives the frequency spectra of the different forms of electric current of FIG. 3;

FIG. 5 shows the power spectra of the different forms of electric current of FIG. 3;

FIG. 6 is a graph comparing the results obtained when. dermis models are stimulated by electric currents having different waveforms;

FIG. 7 is a graph comparing the results obtained when dennis models are stimulated by electric currents having different frequencies;

FIG. 8 is a graph comparing the results obtained when dermis models are stimulated by respectively biphasic and cyclic alternating electric currents;

FIG. 9 shows an exemplary electric current generated by a device according to the invention; and

FIG. 10 shows an exemplary elementary pattern.

### DETAILED DESCRIPTION

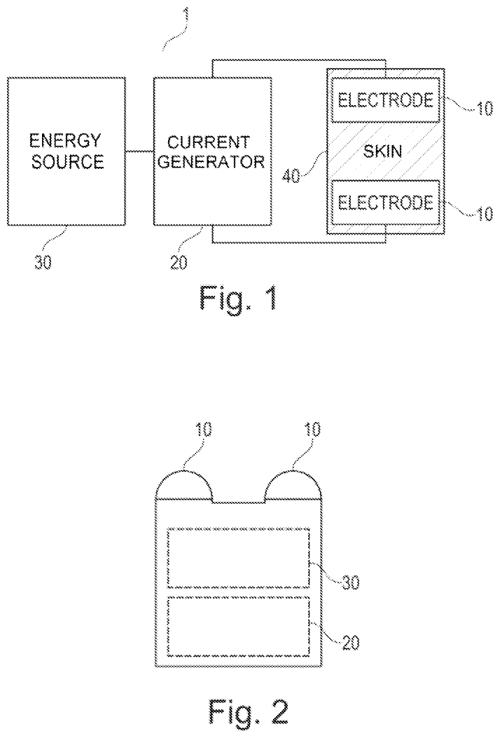

The device 1 according to the invention shown in FIG. 1 comprises a current generator 20 supplied by an electrical energy source 30. This generator is connected to at least two electrodes 10 to be applied to the skin. The generator is configured to generate a sawtooth biphasic or cyclic alternating electric current.

The electrical energy source 30 may comprise at least one battery or cell.

Preferably, the current generator 20 is a low-frequency current generator, operating for example at a frequency of about 100 Hz.

The current generator 20 may comprise a polarity inverter, operating in particular periodically.

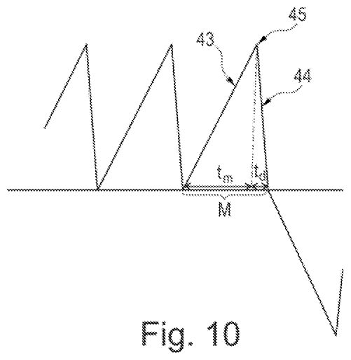

A current generated according to the invention reproduces a succession of elementary patterns M, each composed of a substantially monotonic variation of the electric current in one direction followed by a substantially monotonic variation of the electric current in the opposite direction. An exemplary elementary pattern M is shown in FIG. 10. In FIG. 10 it is possible to see a variation in the electric current in one direction 43, also called a rising edge, followed by a variation in the opposite direction 44, also called a falling edge. The rising edge exhibits an increase in the intensity in terms of absolute value, whereas the falling edge exhibits a decrease in the intensity in terms of absolute value. At point 45, a discontinuity between the rising edge and the falling edge can be seen. The rising edge extends over a duration tin and the falling edge over a duration td. In this example, the rising edge extends over a longer duration than the falling edge.

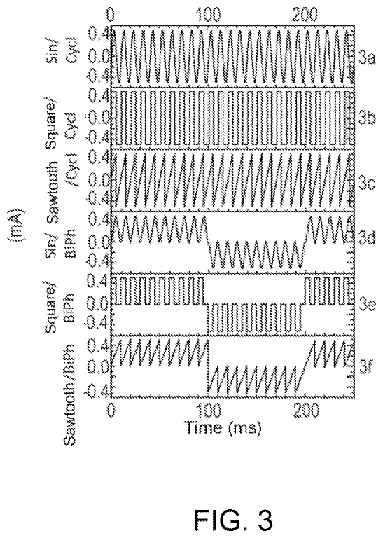

FIGS. 3c, 3f and 9 show exemplary waveforms of electric currents generated by a device according to the invention.

Cyclic Alternating

The current generator 20 may be configured to deliver a sawtooth cyclic alternating electric current, as shown in the example of FIG. 3c.

It can be seen in FIG. 3c that the electric current shown has a constant peak intensity. Preferably, the peak intensity is between 0.1 mA and 2.5 mA, more preferably between 0.3 mA and 1.5 mA, even more preferably about 0.5 mA, as shown in the example of FIG. 3c.

An elementary pattern M of the electric current of FIG. 3c extends over a duration of 5 ms. Preferably, this duration is between 1 ms and 100 ms, more preferably between 2 ms and 20 ms. A cycle comprising an elementary pattern of a certain polarity followed by an elementary pattern of opposite polarity may extend over a duration of about 10 ms.

Additionally, in this example, the elementary patterns M of a certain polarity are not symmetrical with respect to the elementary patterns of opposite polarity. The rising edge 43 of positive polarity does not have the same slope as the rising edge 43′ of negative polarity. The rising edge 43 of positive polarity has a slope that is less steep than the slope of the rising edge 43′ of negative polarity. Similarly, the falling edge 44 of positive polarity does not have the same slope as the falling edge 44′ of negative polarity, the slope of the falling edge 44 of positive polarity being steeper than the slope of the falling edge 44′ of negative polarity.

Biphasic

The current generator 20 may be configured to deliver a sawtooth biphasic electric current, as shown in the examples of FIGS. 3f and 9.

The exemplary electric currents according to the invention shown in FIG. 3f and FIG. 9 have a constant peak intensity. Preferably, the peak intensity is between 0.1 mA and 2.5 mA, more preferably between 0.3 mA and 1.5 mA, even more preferably about 0.5 mA, as shown in the examples of FIGS. 3c, 3f and 9.

In the examples of FIGS. 3f and 9, each elementary pattern M extends over a duration of 10 ms. Additionally, in these examples, each cycle 41; 41′ of positive and negative polarity comprises ten elementary patterns M.

In the examples of FIGS. 3f and 9, the elementary patterns M of a certain polarity are not symmetrical with respect to the elementary patterns of opposite polarity. Specifically, the rising edge 43 of positive polarity does not have the same slope as the rising edge 43′ of negative polarity. Similarly, the falling edge 44 of positive polarity does not have the same slope as the falling edge 44′ of negative polarity. In these particular examples, the rising edge 43 of positive polarity has a slope that is substantially equal to the slope of the falling edge 44′ of negative polarity and the rising edge 43′ of negative polarity has a slope that is substantially equal to the slope of the falling edge 44 of positive polarity.

In the examples shown in FIGS. 3f and 9, it can be seen that each cycle 41 or 41′ comprises the same number of elementary patterns M. The rising edge 43 of an elementary pattern M of positive polarity has a slope, |dI/dt|, that is less than the slope of the falling edge 44 of the elementary pattern M of positive polarity. Conversely, the rising edge 43′ of an elementary pattern M of negative polarity has a slope, |dI/dt|, that is greater than the slope of the falling edge 44′ of the elementary pattern M of negative polarity.

Equivalently, the rising edge 43 of an elementary pattern of positive polarity may have a slope that is greater than the slope of the falling edge 44 of the elementary pattern of positive polarity and the rising edge 43′ of an elementary pattern of negative polarity may have a slope that is less than the slope of the falling edge 44′ of the elementary pattern of negative polarity.

In the example of FIG. 3f, the rising edge 43 of positive polarity has a slope, |dI/dt|, that is approximately equal to 0.05 A/s, and the rising edge 43′ of negative polarity has a slope, |dI/dt|, that is approximately equal to 2.1 A/s. Conversely, the falling edge 44 of positive polarity has a slope, |dI/dt|, that is approximately equal to 2.1 A/s, and the falling edge 44′ of negative polarity has a slope, |dI/dt|, that is approximately equal to 0.05 A/s.

Gel

A conductive gel is preferably applied to the region of skin to be treated, allowing the resistance of the interface with respect to that of the skin to be minimized. Thus, the electric current can flow between the electrodes substantially without modification of its amplitude due to a variation in the quality of th.e interface. Th.e voltage of the alternating current generator 20 is preferably adjusted in real time in order to maintain the intensity at the desired value. Thus, the voltage may vary in order to adapt to the presence or otherwise of the conductive gel on the skin, the moisture level of the skin and/or the skin region treated. This advantageously makes it possible to take into account the sensitivity of the skin region treated and to avoid any painful sensations when using the device, for example sensations of tingling and/or irritation,

Housing

The device may comprise a user interface allowing adjustments to be made, in particular to adjust the voltage of the alternating current generator 20, and/or define and/or view a duration of application of the cosmetic or dermatological treatment,

The device may make it possible to select predefined voltages according to the presence of a gel, or the skin region treated.

FIG. 2 shows an exemplary housing according to the invention. The electrodes 10 are borne by the housing accommodating the electrical energy source 30, and the current generator 20.

The electrodes have an area of contact with the skin that is rounded in shape, and may be free to rotate, the area of contact with the skin being greater than or equal to 1 cm². The electrodes are each, for example, in the form of a ball.

Comparison of the Effects of Stimulation for Different Electric Currents

FIG. 3 shows different forms of electric current. FIGS. 3a and 3b show cyclic alternating electric currents, outside the scope of the invention, respectively of sinusoidal and square waveform. FIG. 3c shows a cyclic alternating electric current, according to the invention, of sawtooth waveform. FIGS. 3d and 3e show biphasic electric currents, outside the scope of the invention, comprising 10 elementary patterns per cycle, respectively of sinusoidal and square waveform. FIG. 3f shows a biphasic electric current, according to the invention, comprising 10 elementary patterns per cycle, of sawtooth waveform.

In Vitro Stimulation Protocol

Dermis models are distributed. in wells of a culture plate. Each well contains a dermis model, made from 3.5 ml of a solution containing 1 to 1.5 mg/ml bovine collagen and 500 000 human fibroblasts, cultured in MEM (minimal essential medium) with 10% FCS, L-glutamine, non-essential amino acids. Na-pyruvate and antibiotics.

The wells are covered with a cover provided with electrodes made of carbon, such that the electrodes penetrate into the dermis solution, allowing the electric current to flow through the dermis models.

The electric current is generated by a current generator, for example the Keysight B2911A current generator, allowing different. waveforms, in particular sawtooth, square, and sinusoidal, different frequencies, and different cycles to be set.

Each dermis model is then subjected to an electric current of peak intensity equal to 0.5 mA for 4 h. Other dermis models are set up in the same way, with the electrodes in contact, but are not subjected to an electric current, thereby making it possible to compare the results of the electrical stimulations on the dermis models with respect. to a situation without electrical stimulation. These dermis models which are not electrically stimulated are hereinafter referred to as the “reference dermis model”.

Comparison of Cyclic Alternating/Biphasic

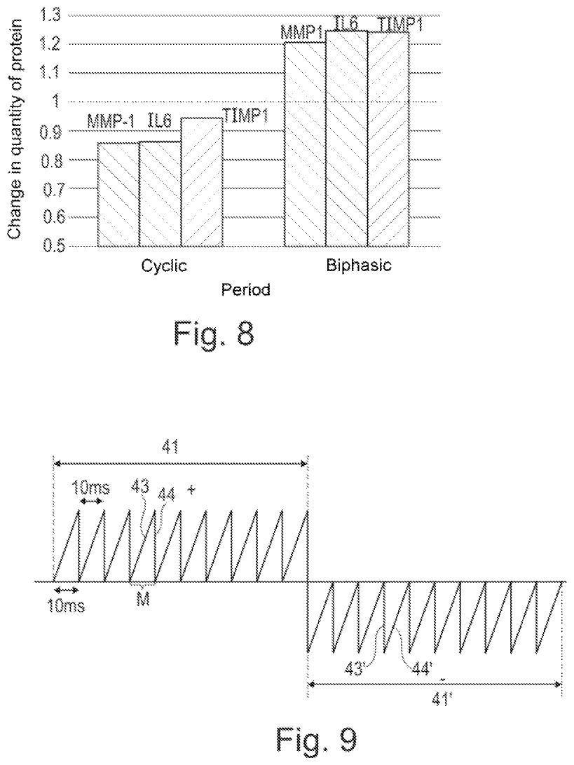

FIG. 8 shows a graph illustrating the effect of electrical stimulation of a dermis model using a sawtooth biphasic electric current comprising cycles of ten elementary patterns and the effect of electrical stimulation of a dermis model using a sawtooth cyclic alternating electric current.

The frequency of the biphasic and cyclic alternating electric currents is 100 Hz.

The electrical stimulation of a dermis model using a biphasic electric current simultaneously promotes an increase in the quantity of the proteins IL6, TIMP1 and of MMP1. Specifically, more IL6, TIMP1 and MMP1 is observed in the dermis model stimulated by the biphasic current than in a reference dermis model (see FIG. 8).

IL6 is a cytokine released by fibroblasts. TIMP1 is a natural inhibitor of MMPs such as MMP1, MMPs allowing in particular the remodeling of dermal tissues by breaking down components of the extracellular matrix. In particular, IL6 and TIMP1 allow the regulation of MMP1. Fibronectins are proteins present in the extracellular matrix, promoting in particular the adhesion of cells to the extracellular matrix and the assembly of fibrillar collagen fibrils formed of type 1 collagen.

The increase in IL6, TIMP1, MMP1 and fibronectins is an indicator thvorable to the remodeling of the skin.

In contrast to the stimulation using a biphasic electric current, a decrease in the quantity of TIMP1, MMP1 and IL6 is observed in the dermis model stimulated using a cyclic alternating electric current with respect to the amount of TIMP1, MMP1 and IL6 present in a reference dermis model.

The electric current generated according to the invention is therefore preferably biphasic.

Comparison of the Effect of the Waveform

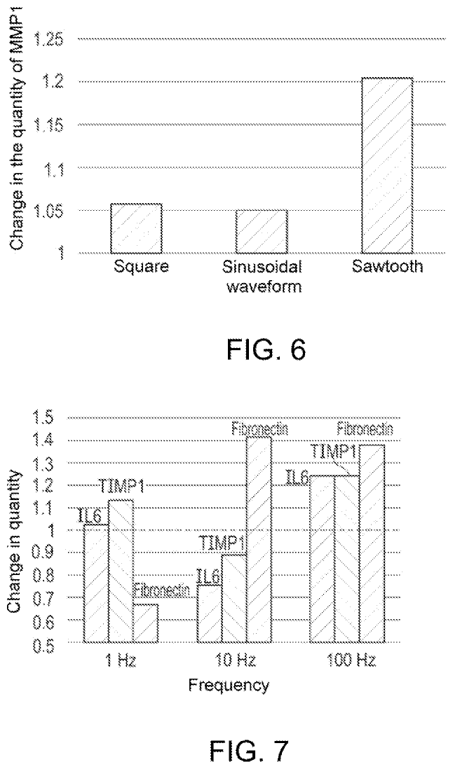

FIG. 6 shows a graph illustrating the effect of electrical stimulation of a dermis model using a biphasic electric current having a sawtooth waveform, according to the invention, the effect of electrical stimulation of a dermis model using an electric current having a square waveform, and the effect of electrical stimulation of a dermis model using an electric current having a sinusoidal waveform.

The frequency of the electric currents generated is 100 Hz.

The peak intensity of the electric currents generated is 0.5 mA.

In general, an increase in the quantity of MMPI secreted by the fibroblasts is observed during electrical stimulation of a dermis model using an electric current with. respect to the quantity of MMPI present in a reference dermis model.

However, stimulation of a dermis model using an electric current having a sawtooth waveform allows a greater increase in the quantity of MMPI with respect to the electric currents having a square and/or sinusoidal waveform. A sawtooth waveform is significantly more effective.

Effect of Frequency

FIG. 7 shows a graph illustrating the effect of electrical stimulation of a dermis model using an electric current having a frequency of 1 Hz, of 10 Hz and of 100 Hz.

The electric currents generated have a biphasic sawtooth waveform.

The stimulation of a dermis model using a 1 Hz electric current has no effect on the quantity of IL6 and TIMP1 with respect to a reference dermis model.

The stimulation of a dermis model using a 10 Hz electric current promotes an increase in the quantity of fibronectin with respect to a reference dermis model.

The stimulation of a dermis model using a 100 Hz electric current promotes an increase in the quantity of IL6, TIMP1 and fibronectin with respect to a reference dermis model.

Additionally, it is advantageous that the level of modulation of TIMP1 is similar to that of MMP1.

Thus, a device according to the invention, generating an electric current with a frequency of about 100 Hz, promotes the stimulation of the skin, allowing the remodeling thereof.

Additionally, electrical stimulation of the skin using an electric current having a sawtooth waveform, a frequency of about 100 Hz and being biphasic also allows an increase α-SMA, a protein of the cytoskeleton. present in the fibroblasts which actively remodel the extracellular matrix such as in the phases of wound healing. When the expression of α-SMA is transient and controlled in the fibroblasts, it is beneficial to the renewal of the matrix. For example, levels of a-SMA are high in the ventral skin of females post-partum (in the weeks following the birth of their babies).

Electrical stimulation of the skin using an electric current having a sawtooth waveform, a frequency of about 100 Hz and being biphasic surprisingly makes it possible to obtain biological responses that are more advantageous than electrical stimulation of the skin. using an electric current having a square or sinusoidal waveform and/or a frequency of 1 Hz or 10 Hz and/or being cyclic alternating.

The differences between the electric currents studied and shown in FIG. 3 are more explicit when these electric currents are observed in the frequency domain.

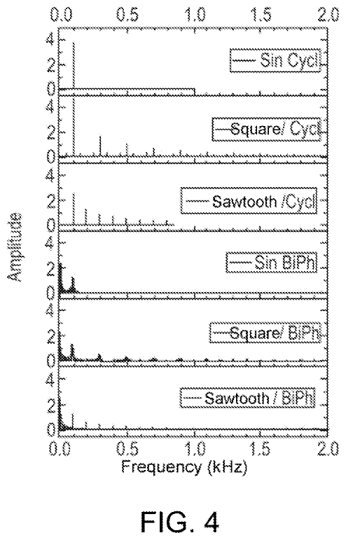

FIG. 4 shows the frequency spectra of the electric currents shown in FIG. 3.

It can be seen that the distribution of the harmonics is highly distinct when the three, square, sinusoidal and sawtooth, waveforms are compared for each of the two, cyclic alternating and biphasic, modes.

The harmonics of the cyclic alternating electric currents are spaced further apart than those of the biphasic electric currents.

More low-frequency harmonics are also observed to be present in the frequency representation of the biphasic electric currents than in the frequency representation of the cyclic alternating electric currents.

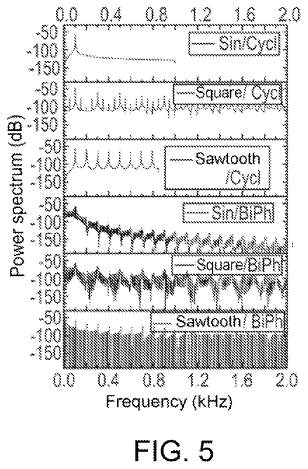

FIG. 5 shows the power spectra of the electric currents of FIG. 3. The difference between these different electric currents is even more explicit when the power spectra are observed.

The invention is not limited to the embodiments described and shown above. Thus, the current may be applied using a plurality of electrodes. The frequency may vary within a cycle, as may the amplitude, and the slope of the rising and falling edges. The rising and falling edges may be non-linear.

1. A device for electrically stimulating skin, comprising:
an electronic circuit for generating a sawtooth biphasic or cyclic alternating electric current;
at least two electrodes connected to the electronic circuit, which are configured so as to be applied to the skin in order to allow the electric current generated to pass therethrough.

2. The device as claimed in claim 1, the electric current having a frequency of between 1 Hz and 1000 Hz and/or the electric current being biphasic and/or the current generated being asymmetric and/or the electric current being biphasic, a cycle of a certain polarity comprising the same number of elementary patterns as a cycle of opposite polarity and/or the electric current being biphasic. a cycle comprising between 3 and 100 elementary patterns.

3-6. (canceled)

7. The device as claimed in claim 1, the device being configured such that a falling edge of an elementary pattern or that a rising edge of an elementary pattern extends over a duration of between 1/2000 and ½ of the duration of the elementary pattern. and/or
the device being configured such that a falling edge of an elementary pattern or that a rising edge of an elementary pattern extends over a duration of between ½ and 1999/2000 of the duration of the elementary pattern and/or
the device being configured such that a falling edge of an elementary pattern and/or a rising edge of an elementary pattern comprises a decrease, or respectively an increase, in the intensity of the electric current per second, in terms of absolute value. of between 0.001 A and 2.5 A,
and/or the device being configured such that a falling edge of an elementary pattern and/or a rising edge of an elementary pattern comprises a decrease. or respectively an increase, in the intensity of the electric current per second, in terms of absolute value, of between 0.05 A and 100 A.

8-10. (canceled)

11. The device as claimed in claim 1, the device being configured such that a rising edgeof an elementary pattern extends over a longer duration than a falling edge of the elementary pattern.

12. The device as claimed in claim 1, the peak intensity being constant.

13. The device as claimed in claim ls, the intensity of the current being such that the electric current density delivered by the electrodes is between 0.1 mA/cm² and 2.5 mA/cm².

14. The device as claimed in claim 1, the electronic circuit being arranged such that the electric current has elementary patterns that extend over a duration of between 1 ms and 100 ms and/or the electronic circuit being configured such that the voltage between the electrodes without load is between 0.01 V and 45 V.

15. (canceled)

16. The device as claimed in claim 1, the electrodes being borne by a housing accommodating the electronic circuit, and/or the electrodes being borne by a housing accommodating an energy source and/or the electrodes each having an area of contact with the skin that is greater than or equal to 1 cm² and/or the electrodes having an area of contact with the skin that is rounded in shape and/or the electrodes being to be attached to the skin.

17. (canceled)

18. The device as claimed in claim 1, comprising a conductive gel to be applied to the skin in the region of application of the electrodes and/or comprising a conductive gel to be applied to the skin in the region of application of the electrodes the gel being without a cosmetic active agent such as for example humectant or moisturizing active agents, anti-aging active agents. for example depiumenting active agents, active agents acting on skin microcirculation or seboregulating active agents, in particular vitamin C and derivatives thereof, hyaluronic acid, or ellagic acid.

19-22. (canceled)

23. The device as claimed in claim 1, being arranged so as to take at least one electrical measurement on the skin via said electrodes, before electrically stimulating the skin.

24. A method for non-therapeutically cosmetically treating a healthy skin, comprising the step of subjecting the skin to a sawtooth biphasic or cyclic alternating electric current, delivered by a device as claimed in claim 1.

25. The method as claimed in claim 24, comprising the movement of the electrodes during the treatment.

26. The method as claimed in claim 24, the electric current being applied in the presence of a conductive gel that is without a cosmetic active agent such as humectant or moisturizing active agents, anti-aging active agents, for example depigmenting active agents, active agents acting on skin microcirculation or seboregulating active agents, in particular vitamin C and derivatives thereof, hyaluronic acid, or ellagic acid.

27. The method as claimed in claim 24, the skin region treated exhibiting a lack of firmness, tone or elasticity or comprising a wrinkle.

28. The method as claimed in claim 24, comprising the analysis, by a connected system, of information relating to the skin, originating from electrical measurements taken with the electrodes.
